(19) United States
(12) Patent Application Publication
(10) Pub. No.: US 2017/0205402 A1
(43) Pub. Date: Jul. 20, 2017

(54) IN PLANTA LEAF BIOENSOR

(71) Applicant: The Regents of the University of California, Oakland, CA (US)

(72) Inventors: Hideaki Tsutsui, Alhambra, CA (US); Jessica Wen, Villa Park, CA (US); Carlos Castro, Riverside, CA (US)

(73) Assignee: The Regents of the University of California, Oakland, CA (US)

(21) Appl. No.: 15/326,913

(22) PCT Filed: Jul. 14, 2015
(86) PCT No.: PCT/US15/40452

§ 371 Date: Jan. 17, 2017

Related U.S. Application Data
(60) Provisional application No. 62/024,076, filed on Jul. 14, 2014.

(51) Int. Cl. G01N 33/543; G01N 33/50

(52) U.S. Cl. CPC G01N 33/5432; G01N 33/5097

(57) ABSTRACT

A method for detecting an analyte in a plant is provided. The method includes injecting an analyte-capture agent into a leaf of a plant, and in the leaf, optically detecting complexes formed by capturing an analyte with the injected analyte-capture agent. The analyte-capture agent can include an analyte-interacting component connected to a microcarrier. In particular versions, the analyte-capture agent can be an analyte specific antibody or aptamer, and the microcarrier can be a polydiacetylene liposome. Binding of the analyte can cause a change in color of the liposome, thus indicating the presence of the analyte.

### CROSS-REFERENCE TO RELATED APPLICATIONS

This application claims the benefit of U.S. Provisional Patent Application No. 62/024,076, filed on Jul. 14, 2014, which is incorporated by reference herein.

### BACKGROUND

Field of the Invention

The invention relates to methods of detecting analytes in plants.

Related Art

Quick and efficient detection of plant pathogens is desirable for the timely intervention of plant diseases. Low-cost and quick detection of biotic stresses in plants is critically important for protection of staple food crops such as maize in smallholder farms in the developing countries, where access to improved seed varieties, fertilizers and pesticides is limited due to financial and geographical reasons. Standard methodologies in plant diagnostics range from physical examination of symptoms to the use of molecular diagnostics such as enzyme-linked immunosorbent assay (ELISA) or polymerase chain reaction (PCR) [4-6]. ELISA and PCR offer high sensitivity and specificity; however, both these approaches require expensive laboratory facilities and trained operators. On the other hand, physical examination can only offer a diagnosis well past the onset of pathogenic infection.

Maize is one of the most widely grown staple crops globally, and is especially critical in developing regions of sub-Saharan Africa and Mesoamerica, where maize alone comprises over 20% of food calories and up to 73% of total production is used as a food source [1]. Despite high demand, maize growth suffers from significant losses at both pre- and post-harvest stages due to biotic stresses such as viruses, fungi, bacteria, and other pests and pathogens. Numerous methods to improve maize growth and yield, including adoption of improved seed varieties, use of fertilizers, and application of pesticides, have been suggested to alleviate recurrent food shortages; however, these have not been widely employed by smallholder farmers due to their high cost and lack of accessibility [2,3].

Lateral flow biosensors can be used as diagnostic tools. A common example of a lateral flow biosensor is the home pregnancy test, where human chorionic gonadotropin in urine is detected by preprinted capture and detector antibodies conjugated with colloidal gold particles, forming visible lines on a paper substrate. Recently, lateral flow devices have been developed to detect plant pathogens [5] and have gained much attention as inexpensive, user-friendly, in-field diagnostic tools [7]. However, these external devices require the user to initiate testing and are likely used for rapid confirmation of pathogenic infection already suspected from physical examination. Lateral flow devices typically incorporate the use of a nitrocellulose test strip and capture antibodies to detect the presence of analyte in a two-step sandwich assay. Wicking of analyte-containing solution onto the nitrocellulose substrate results in capture of analyte by the first antibody, conjugated to a colored particle. The analyte-antibody pair then flows downstream to a second analyte-specific antibody that is immobilized on the surface of the test strip (the detection zone). The accumulation of the initial analyte-antibody pairs at the detection zone results in a positive test indicated by the aggregation of colored particles visible to the naked eye [7].

Syringe agroinfiltration is a method of gene introduction into plant hosts via delivery of genes through Agrobacterium [8]. A needleless syringe is filled with Agrobacterium-containing media. The tip of the needleless syringe is then placed against the abaxial (back-side) of an intact plant leaf and the media is manually injected into the leaf interstitium. A temporary color change from light green to dark green indicates successful infiltration of Agrobacterium into the leaf [8,9]. Syringe agroinfiltration has been demonstrated successfully in several plant species [10,11]. Other common methods of transient gene expression in live plant hosts include biolistics (microprojectile bombardment) [12] and microneedle injection [13]; however, the need for minimal equipment and simplicity of procedure has made syringe agroinfiltration a popular choice in recent years.

### SUMMARY

In one aspect, a method for detecting an analyte in a plant is provided. The method includes injecting an analyte-capture agent into a leaf of a plant, and in the leaf, optically detecting complexes formed by capturing an analyte with the injected analyte-capture agent.

In embodiments of the method: a) the analyte-capture agent can include an analyte-interacting component connected to a microcarrier; b) the analyte-interacting component can be an antibody, peptide aptamer, or oligonucleotide aptamer; c) the analyte-interacting component can specifically recognize the analyte; d) the microcarrier can be a microsphere or a vesicle; e) the microcarrier can be a polydiacetylene liposome; f) the analyte can be a metabolite or a nutrient of the plant, or can be a plant pathogen or a component of the plant pathogen, or an environmental toxin; g) the injecting can be performed by propelling the analyte-capture agent into the leaf interstitial space; h) the optically detecting can include colorimetric detection of the complexes after analyte capture by the injected analyte-capture agent; i) the optically detecting can include interacting the complexes with primary and/or secondary antibodies to form antibody antibody combinations, and colorimetric detection of the combinations; j) the plant can be a crop plan; k) the plant can be a living plant, and the leaf can be attached to the plant at least until the analyte capturing; l) the detecting can be performed on chlorotic leaf tissue; or m) any combination of a)-l).

In another aspect, a method for detecting an analyte in a plant is provided. The method includes: injecting an analyte-capture agent into a leaf of a plant, the analyte-capture agent comprising an analyte-interacting component connected to a polydiacetylene liposome, where the analyte-interacting component specifically recognizes an analyte in the plant; forming complexes in the leaf by capturing the analyte with the injected analyte-capturing agent; and in the leaf, visually detecting a color change in the complexes after analyte capture by the injected analyte-capture agent.

In embodiments of the method: a) the analyte-interacting component can be an antibody, peptide aptamer, oligonucleotide aptamer, or protein; b) the analyte can be a pathogen of the plant, or can be a component of the plant pathogen, a plant nutrient, protein, metabolite, or any environmental toxin or foreign compound; c) the plant can be a living plant, and the leaf can be attached to the plant at least until the analyte capture; d) the plant can be a crop plant; e) the detecting can be performed on chlorotic leaf tissue; or f) any combination of a)-e).

### BRIEF DESCRIPTION OF THE DRAWINGS

For a more complete understanding of the present invention, reference is now made to the following descriptions taken in conjunction with the accompanying drawings, in which:

FIGS. 1A-D show methods of one-step lateral flow detection of pathogen markers in live maize leaves.

FIGS. 2A-C show, respectively, optical images, fluorescence intensity and microsphere retention rate after 72 hours.

FIGS. 3A-C show lateral flow detection of fluorescein in live maize plants by: (FIG. 3A) the immobilization of anti-fluorescein antibody onto red fluorescent microspheres, (FIG. 3B) the infiltration of anti-fluorescein coated red fluorescent microspheres in leaf followed by antibody capture of fluorescein of varying concentrations, and (FIG. 3C) the relative green fluorescence intensity of fluorescein captured by anti-fluorescein conjugated microspheres and control (non-coated) microspheres.

FIGS. 4A-C show PDA liposomes. Blue to red color transitions are observed in PDA liposome solutions with both increasing temperature (FIG. 4A) and pH (FIG. 4B). Additionally, thrombin aptamer-conjugated liposomes exhibit significant color transitions in 3 μM thrombin while minimal changes are seen in BSA and FBS solutions (FIG. 4C).

FIG. 5 is a graph showing PDA liposome size controlled by sonication time.

FIGS. 6A-B are images showing the visibility of colored bioreagents in leaves.

FIG. 7 is an image showing enhanced visibility of injected liposomes by leaf patterning through a mask.

### DETAILED DESCRIPTION

Embodiments of the present invention can provide notification at the first onset of pathogenic infection using a biodetection strategy that is integrated directly within the host plant itself. In accordance with some embodiments, a new lateral flow detection technology is provided that is directly integrated in a leaf of a plant such as a flowering plant, where an analyte-capture agent, alternatively called an analyte-specific reagent, is injected in a minimally invasive manner into the leaf interstitium. The analyte-capture agent can capture and detect analyte in a concentration specific manner.

In various aspects, a method of detecting an analyte in a plant is provided. Examples of analytes include, but are not limited to: plant metabolites such as phosphinothricin acetyltransferase; plant nutrients including nitrogen, phosphorous, potassium, calcium, magnesium, sulfur, boron, chlorine, copper, iron, manganese, molybdenum, zinc, or nickel; plant pathogens and biotic stressors including bacteria, viruses, fungi, or parasites; components of plant pathogens and biotic stressors including their proteins, nucleic acids, polysaccharides, or metabolites; and other analytes including toxins such as lead, cadmium, or mercury.

Examples of particular analytes, include but are not limited to: fluorescent analytes such as aflatoxins, riboflavin, or pyridoxine; pathogens such as geminiviruses including maize streak virus, tomato golden mosaic virus, and others such as tomato spotted wilt virus, tomato chlorotic spot virus, groundnut ring spot virus, or chrysanthemum stem necrosis virus; and other analytes.

In the method, the analyte-capture agent can comprise an analyte-interacting component connected to a microcarrier. Examples of microcarriers include, but are not limited to, microparticles, microcapsules, microspheres, polymer particles, vesicles and liposomes, hydrogel capsules, or electrospun particles. A microcarrier can be made from materials such as, but not limited to, styrene, melamine, polyacrylonitrile, polymethylmethacrylate, a lipid polymer, polyethylene glycol diacrylate, or alginate.

In particular embodiments, the microcarrier is a polydiacetylene (PDA) liposome. PDA is a class of lipid polymers with highly conjugated backbones that have unique optical and chromatic properties. PDA materials provide a number of advantages attributable to their ease of formation under self-assembly systems. Additionally, PDA polymerization commonly occurs via photoreaction, allowing for high purity during synthesis by eliminating the problem of un-wanted byproducts from chemical initiator. The chromatic properties of PDA arise from its highly structured, alternating ene-yne backbone. Upon UV or γ-irradiation, PDA polymerizes via 1,4 addition of diacetylene (DA) moiety to appear optically blue and non-fluorescent. Exposing blue PDA to environmental changes results in a shift in absorption spectra from low to high energy resulting in the appearance of optically red PDA that is also fluorescent. During this transition, an intermediate purple phase is observed. PDA materials have been shown to undergo blue to red optical transitions in response to various environmental perturbations including changes in pH, temperature (thermochromism), and molecular binding events (affinochromism/biochromism). The color transitions of traditional PDA materials are irreversible, although studies have demonstrated the synthesis of modified, reversible PDAs. Examples of DAs include, but are not limited to, 10, 12 tricosadiynoic acid (TCDA), or 10, 12 pentacosadiynoic acid (PCDA). Diacetylenes are commercially available from companies such as GFS Chemicals (Powell, Ohio, USA); Sigma-Aldrich (St. Louis, Mo., USA); Alfa Aesar (Ward Hill, Mass., USA).

The microcarrier can be any size that allows the analyte-capture agent to be injected into, and retained in, the leaf interstitial space. In embodiments comprising microspheres, the microsphere can have a diameter in the range of about 0.1 μm to about 1 μm, depending on the plant, and more particularly, can have a diameter of about 0.5 μm. Similar diameter sizes are suitable for vesicles and liposomes used as microcarriers.

The analyte-interacting component can be a molecule or moiety that interacts with, or binds to, an analyte of interest. For example, the analyte-interacting component can be, but is not limited to, an antibody, a peptide aptamer, an oligonucleotide aptamer, a receptor for the analyte, an analyte binding protein or compound, the analyte-interacting portion of such components, or any combination thereof.

The analyte-interacting component and the analyte-capture agent can be specific for the analyte of interest. A component or agent is specific for the analyte of interest, or can specifically recognize the analyte of interest, when the component or agent recognizes the target analyte to a greater degree in comparison to other analytes.

In the method, the plant can be any plant that contains leaves, including a crop plant, an ornamental plant, a fruit or nut bearing tree, or a plant used as feedstock for cattle or other animals. In particular, the plant can be an angiosperm, which is a fruiting plant that produces seeds within an enclosure, and is divided into the subgroups monocots (or monocotyledons) and dicots (or dicotyledons). Examples of monocots include, but are not limited to, lilies, daffodils, sugarcane, banana, palm, ginger, onions, bamboo, sugar, palm tree, banana tree, grass, and grains such as wheat, maize (corn), rice, and millet. Examples of dicots include, but are not limited to, legumes (pea, beans, lentils, peanuts) daisies, mint, lettuce, tomato and oak.

In particular embodiments, the plant is maize, potato, cassava, bean, pepper, tomato, sugar beet, cotton, grapevine, citrus, alfalfa, apple, avocado, banana, beet, barley, blueberry, raspberry, strawberry, soybean, squash, sugarcane, tobacco or peanut.

The analyte-capture agent can be injected into a leaf of a plant. The injecting can be performed by propelling the analyte-capture agent into the leaf interstitial space. In some embodiments, this is accomplished by injecting the analyte-capture agent through pores in the leaf abaxial epidermis. In some embodiments, the injection can be by a needleless syringe.

Complexes formed when the analyte is captured by the analyte-capture agent can be optically detected. Optical detection includes detecting visible, infrared, fluorescence or ultraviolet light. In some cases, the analyte itself emits light, and the complexes be identified by colorimetric detection in the leaf under proper illumination. Examples of such analytes include, but are not limited to, fluorescent analytes such as aflatoxins, riboflavin, or pyridoxine. In other cases, such as with the use of polydiacetylene liposomes, the microcarrier can undergo a color change when an analyte is captured by the analyte-capture agent, and the complexes can be identified by colorimetric detection as a change in color.

In still another case, an in vivo two-step lateral flow detection system can be used. In the first step, pathogen antibody-coated, fluorescent microspheres that are injected and immobilized in the plant interstitial space (for example, via needleless syringe infiltration) can capture target pathogen antigen. Subsequently, fluorescently tagged secondary antibodies (i.e. with horseradish peroxidase) that are produced by the plant leaf via transfection of plant cells through methods including but not limited to agroinfiltration in the region of the leaf containing the microspheres, will bind to the captured antigen in a sandwich assay. The accumulation of the fluorescently tagged secondary antibodies onto the antigens bound on the microspheres will to form a fluorescence detection signal. The circulating secondary antibodies can also be introduced into the plant through the roots.

In various embodiments, a leaf injected with an analyte-capture agent can be attached to, and thus be part of, a living plant. The optical detecting can be performed on an injected leaf that is still attached to the living plant, or can be performed on an injected leaf that is separated from the living plant after the analyte is captured by the analyte-capture agent. In other embodiments, a leaf can be separated from a plant before injection of the analyte-capture agent, or an injected leaf can be separated from a plant prior to analyte capture, for example, where an analyte is present at sufficient concentration in the leaf.

In particular embodiments, a method is provided for detecting biotic stress in a plant comprising injecting a reagent directly into the leaf of a plant for lateral flow detection of analyte associated with the biotic stress. The plant can be a monocot or a dicot, and in particular embodiments is maize. Injection can be by a needleless syringe through pores in the leaf abaxial epidermis to propel the reagent into the leaf interstitial space. More specifically, the microspheres can have a diameter in the range of 0.1 μm to 1 μm, preferably about 0.5 μm. The analyte can be detected by targeting a fluorescent biomarker or by the incorporation with the reagent of detection-sensitive, stimuli-responsive colorimetric polydiacetylene vesicles. This in-plant lateral flow biosensor is the first of its kind and can be expected to provide a low-cost and user-friendly detection method for biotic stresses in the field.

In some embodiments, the analyte-capture agent can be composed of, for example, antibody-conjugated microspheres, or the analyte-capture agent can be formed of microspheres conjugated with oligonucleotides capable of specifically binding with high affinity to non-nucleotide target molecules. Such nucleic acids are commonly referred to as aptamers and can be, for example, an RNA or a DNA oligonucleotide composed of naturally occurring and/or modified nucleotides. DNA aptamers are frequently used as alternatives to antibodies in research. They are cheaper and chemically more stable, are specific to target antigens, and are less sensitive to the chemistry required to attach to the microspheres. Alternatively, peptide aptamers and/or proteins can be used as part of the analyte-capture agent.

An antibody is an immunologic binding agent such as IgG, IgM, IgA, IgD and IgE. Techniques for preparing and using various antibody-based constructs and fragments are well known in the art. Means for preparing and characterizing antibodies are also well known in the art (See, for example, Harlow and Lane, “Antibodies: A Laboratory Manual,” Cold Spring Harbor Laboratory, 1988, incorporated by reference herein). Monoclonal antibodies (mAbs) are recognized to have certain advantages, e.g., reproducibility and large-scale production. Thus, monoclonal antibodies of the human, murine, monkey, rat, hamster, rabbit and even chicken origin, are contemplated for use. In some embodiments, an antibody-like molecule that has an antigen binding region may be appropriate. Examples of such anti-body like molecules include, but are not limited to, antibody fragments such as Fab′, Fab, F(ab′)2, single domain antibodies (DABs), Fv, scFv (single chain Fv), and the like. Antibodies against plant pathogens are available from commercial sources such as Agdia (Santa Fe Springs, Calif., USA) and Neogen (Lansing, Mich., USA).

An analyte-specific aptamer can be identified by library screening, in which a library of aptamers containing a vast number of oligonucleotide or peptide sequences is screened against the target antigen and the sequence with the highest affinity for the antigen is determined. The chosen sequence is then subject to further mutation or variation to form a new library of aptamers that is further subject to screening against the target antigen. The screening cycles continue until an aptamer with a sequence of sufficiently high affinity and specificity is found. Aptamers against plant pathogens such as geminiviruses, tomato spotted wilt virus, tomato chlorotic spot virus, groundnut ring spot virus and chrysanthemum stem necrosis virus, for example, have been described [17-18].

In particular embodiments, the analyte-capture agent can be a capture antibody that is specific to the analyte, and can be chosen to detect the analyte in a concentration dependent manner. In other embodiments, the analyte-capture agent can be an aptamer that interacts specifically with the analyte, and can be chosen to detect the analyte in a concentration dependent manner

Embodiments of the invention provide the first development of a lateral flow biosensor that utilizes the leaf itself as the substrate for the lateral flow “test strip”. In one embodiment, by modifying the established agroinfiltration procedure capture-antibody-conjugated microspheres are introduced and immobilized in the leaf interstitium. This is a one-step lateral flow detection platform that targets fluorescent biomarkers (e.g. aflatoxins) or incorporates the use of detection-sensitive, colorimetric polydiacetylene (PDA) vesicles [14,15] to simplify the procedure and eliminate the need for two-step sandwich assays. Further in accordance with embodiments of the invention, microsphere size is optimized for effective infiltration and immobilization in the leaf.

The present invention may be better understood by referring to the accompanying examples, which are intended for illustration purposes only and should not in any sense be construed as limiting the scope of the invention.

### Example 1

### Methods and Materials

### Materials

Maize seeds were obtained from Carolina (Burlington, N.C.). 1 mL needleless syringes were from Fisher Scientific (Waltham, Mass.). Fluorescent microspheres were obtained from Spherotech (Lake Forest, Ill.), and biotinylated anti-fluorescein antibody was from eBiosciences (San Diego, Calif.). All other chemicals used are research grade and were obtained from Sigma Aldrich (St. Louis, Mo.).

### Maize Growth

Maize seeds were germinated in coarse vermiculite in a greenhouse maintained within a temperature range of 16-27° C. (61-81° F.). Seeds and plants were immersed once a day in Peters Excel 21-5-20 Multi Purpose fertilizer solution (125 ppm N) from Everris (Dublin, Ohio).

### Infiltration of Microspheres

Fluorescent microspheres were washed in phosphate buffered saline (PBS) solution and resuspended in infiltration buffer (10 mM MgCl₂, 10 mM MES-potassium, pH 5.6). Microsphere solutions (0.5% solids) were infiltrated into the abaxial side of intact maize leaves. Infiltrated leaves were examined under a Leica DM2000 fluorescence microscope immediately following infiltration and every 24 h for 72 h. Fluorescence intensities were determined as a ratio of the signal intensity of the target region to the leaf background. Retention rate is calculated as a ratio of fluorescence intensity at 72 h after infiltration and immediately following infiltration.

### Lateral Flow Detection

Anti-fluorescein antibody-coated red fluorescent microspheres (AF microspheres) were prepared by mixing 0.5 μm streptavidin-coated red fluorescent microspheres with biotinylated anti-fluorescein antibodies, washed, and resuspended in the infiltration buffer. AF microsphere solutions (0.5% solids) were injected into 15-20 day old maize plants, examined for initial microsphere content and transferred to fluorescein solutions (pH=6.8). The plants were grown in fluorescein solution for 72 h, and solution was changed every 12 h. The plants were examined again at the end of the 72 h experiment.

### Results and Discussion

### Infiltration of Microspheres in Leaf Tissue

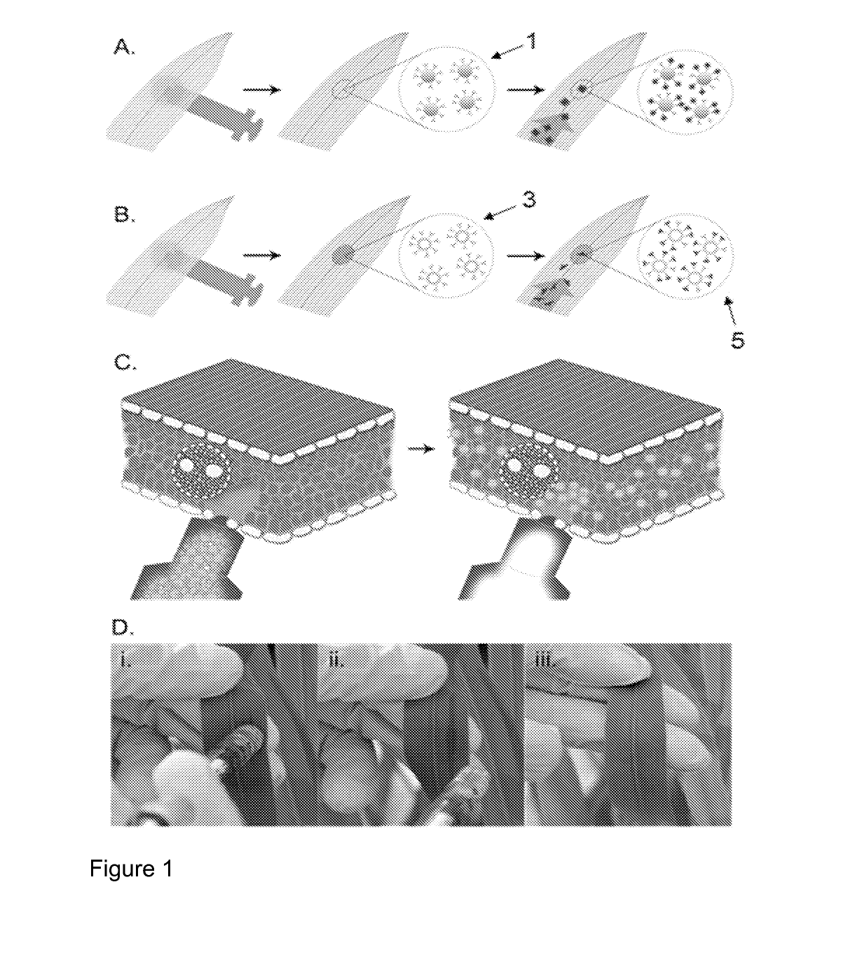

FIG. 1 shows in subfigures A through D methods of one-step lateral flow detection of pathogen markers in live maize leaves. FIG. 1A depicts the detection of fluorescent biomarker using antibody-conjugated microspheres 1, which serves as the basis for detecting non-fluorescent biomarkers by incorporating stimuli-sensitive colorimetric vesicles, as shown in FIG. 1B, in which a color shift (e.g. from blue 3 to red 5) visible to the naked eye is observed upon binding of analyte to antibodies conjugated to the vesicle surface. A pressure-driven infiltration technique was developed to non-invasively introduce microspheres into the leaf. FIG. 1C is a schematic of the infiltration of microspheres into leaf tissue before infiltration (left) and after infiltration (right). A pressure gradient, introduced by the syringe on the leaf surface, propels bioreagents through pores in the leaf abaxial epidermis and into the leaf interstitial space. FIG. 1D shows infiltration in a maize leaf: (i) with a needleless syringe, (ii) immediately after injection, where injected buffer solution is visible, and (iii) 10 min after injection, where injected buffer evaporates without leaving any visible mark. Although agroinfiltration procedures are most commonly performed on Nicotiana benthamiana, as shown in FIG. 1D we successfully demonstrate infiltration in maize leaves without damaging the leaf epidermis. In accordance with this invention, microspheres are used to provide stationary surfaces through which antibodies can be immobilized within the leaf tissue. Without such surfaces, antibodies cannot be retained at a fixed spot inside the leaf.

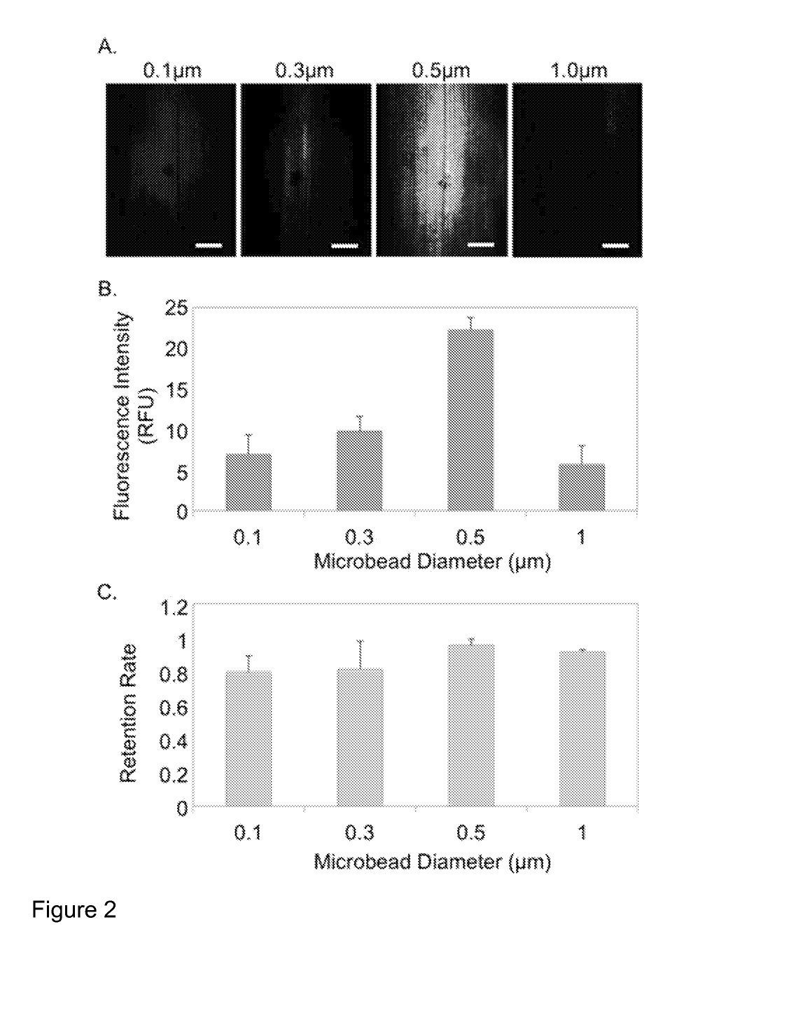

As shown in FIGS. 2A though 2C, microsphere sizes were characterized for both high infiltration efficiency and retention rate post-infiltration. Spheres too large cannot pass through the leaf epidermis, whereas spheres too small are easily removed from the infiltrated spot. Fluorescent microspheres, ranging from 0.1 μm to 1 μm in diameter, were infiltrated into intact maize leaves. FIG. 2A shows optical images of infiltrated microspheres. FIG. 2B shows the fluorescence intensity of infiltrated microspheres by diameter. Microscopy analysis of the microspheres injected and retained at the injection site over the 72 h period indicates that the 0.5 μm diameter microspheres produce initial signal intensities 2.5, 3, and 4 times higher than 0.3, 0.1, and 1.0 μm infiltrated microspheres, respectively. Additionally, as shown in FIG. 2C, 0.5 μm infiltrated microspheres yield a nearly 100% retention rate over 72 h. These results suggest that a 0.5 μm diameter particle size is desirable for high infiltration and retention efficiency, and is appropriate for the immobilization of foreign biomolecules (e.g., antibodies) in the maize leaf tissue.

### In Planta Lateral Flow Detection of Fluorescein by AF Microspheres

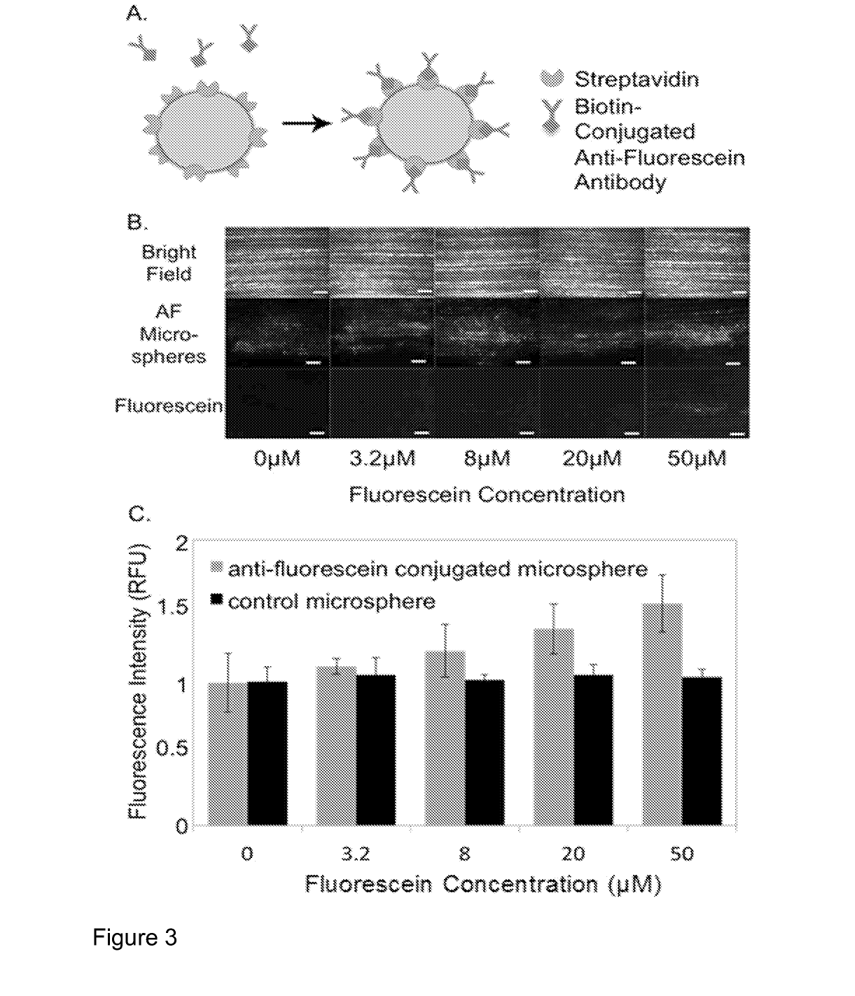

Detection of fluorescein by AF microspheres was performed as a proof-of-concept demonstration of the in planta biosensing technology. FIG. 3 shows lateral flow detection of fluorescein in live maize plants by: (FIG. 3A) the immobilization of anti-fluorescein antibody onto red fluorescent microspheres, (FIG. 3B) the infiltration of anti-fluorescein coated red fluorescent microspheres in leaf followed by antibody capture of fluorescein of varying concentrations, and (FIG. 3C) the relative green fluorescence intensity of fluorescein captured by anti-fluorescein conjugated microspheres and control (non-coated) microspheres. Referring specifically to FIG. 3C, analysis of fluorescein detection by AF microspheres presents a 22%, 34%, 51%, and 71% increase in fluorescence intensity by plants infiltrated with AF microspheres grown in 3.2 μM, 8 μM, 20 μM, and 50 μM fluorescein solution respectively, as compared to control plants grown in 0 μM fluorescein solution. In addition, plants infiltrated with control (non-conjugated) microspheres do not show increased fluorescence intensity with increasing fluorescein concentration, further confirming specific detection by the test plants that are infiltrated with AF microspheres. The limit of detection (LoD) [16] is 19 μM. Collectively, these data indicate the feasibility of in planta detection of biomolecules in maize based on this technology.

### Conclusion

The invention provides a minimally invasive in planta biomolecular detection platform for flowering plants, exemplified by maize. The design of the detection system incorporates the immobilization of antibody-conjugated microspheres in the leaf to capture analyte and produce a detection signal. Unlike conventional lateral flow assays, which require sample processing (e.g. grinding of plant and extraction of analyte solution) and are single-use, the directly integrated technology does not require processing of plants post-infiltration and can continuously monitor biomarker levels for the early notification of disease onset. This not only eliminates the need for multiple tests per plant but also an external substrate for the lateral flow test strip, thereby reducing the costs and resources needed for disease detection. This simple method is minimally-invasive, cost-effective, and is, to the inventors' best knowledge, the first report of a lateral flow biodetection technology that is integrated directly within living plant hosts. Because syringe infiltration is easily translatable [10,11], this technology is applicable for a variety of plants, monocots and dicots alike. With the incorporation of colorimetric, stimuli-responsive PDA vesicles [14,15], this technology can yield an equipment-free and colorimetric in planta diagnosis.

### Example 2

PDA liposomes are biocompatible [22,23], colorimetric sensors that can be surface-modified with detection probes to detect target biomolecules [24,25], making them appropriate as in vivo sensors.

PDA liposomes have been synthesized that respond to temperature and pH changes in solution as well as specific reaction with thrombin. PDA liposomes were prepared by suspending TCDA and PCDA monomers at a total of 3 mM in deionized water followed by sonication at 80° C. (glass transition, Tg˜60° C.). Upon 254 nm UV irradiation, the liposome solution turns from opaque to deep blue.

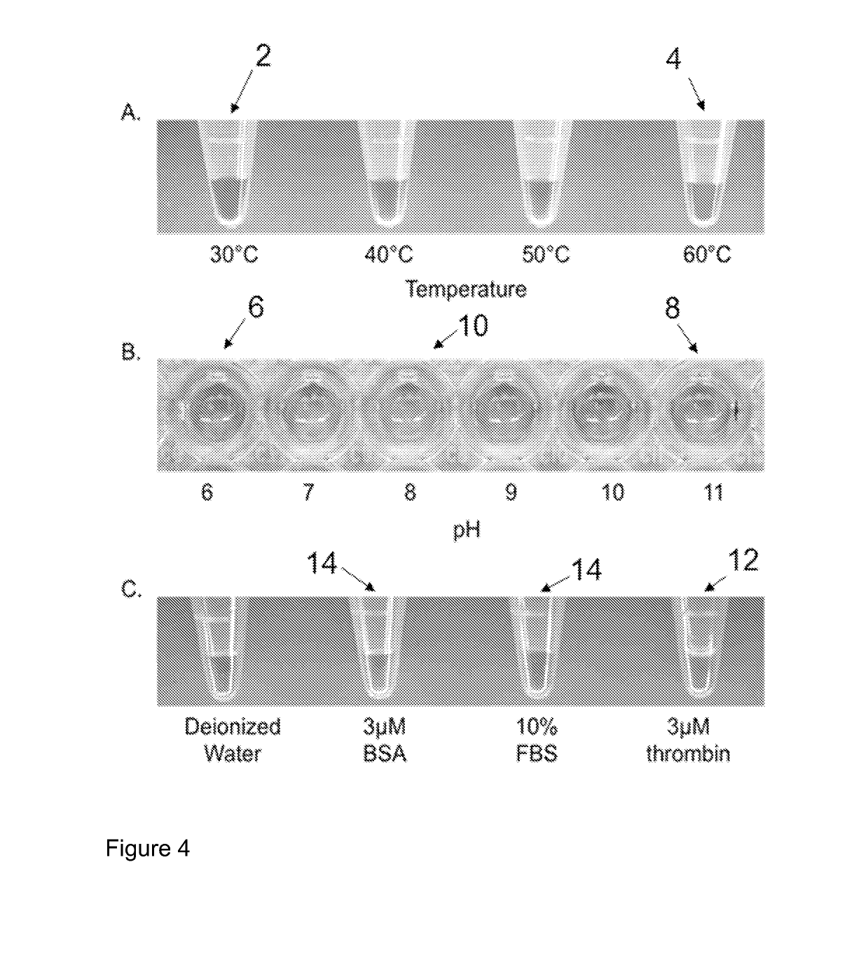

FIGS. 4A and 4B show color changes from blue colored sample 2 to red colored sample 4 based on temperature, and from blue colored sample 6 to red colored sample 8 based on pH. An intermediate purple colored sample 10 is also shown.

Additionally, following a published protocol [26], PDA liposomes for specific thrombin detection were prepared. The thrombin-sensing liposomes were prepared using thrombin aptamer-conjugated TCDA monomers that were synthesized by activating the carboxylic acid moiety of the TCDA with N-(3-dimethylaminopropyl)-N′-ethylcarbodiimide hydrochloride (EDC-HCl) and N-hydroxysuccinimide (NHS). The thrombin-sensing liposomes were composed of 6.7% thrombin aptamer-conjugated TCDA monomers, 53.3% unmodified PCDA monomers, and 40% DMPE. FIG. 4C shows the red colored sample 12 of the liposomes in the presence of thrombin compared to the blue colored samples 14 in the presence of bovine serum albumin (BSA) or fetal bovine serum (FBS).

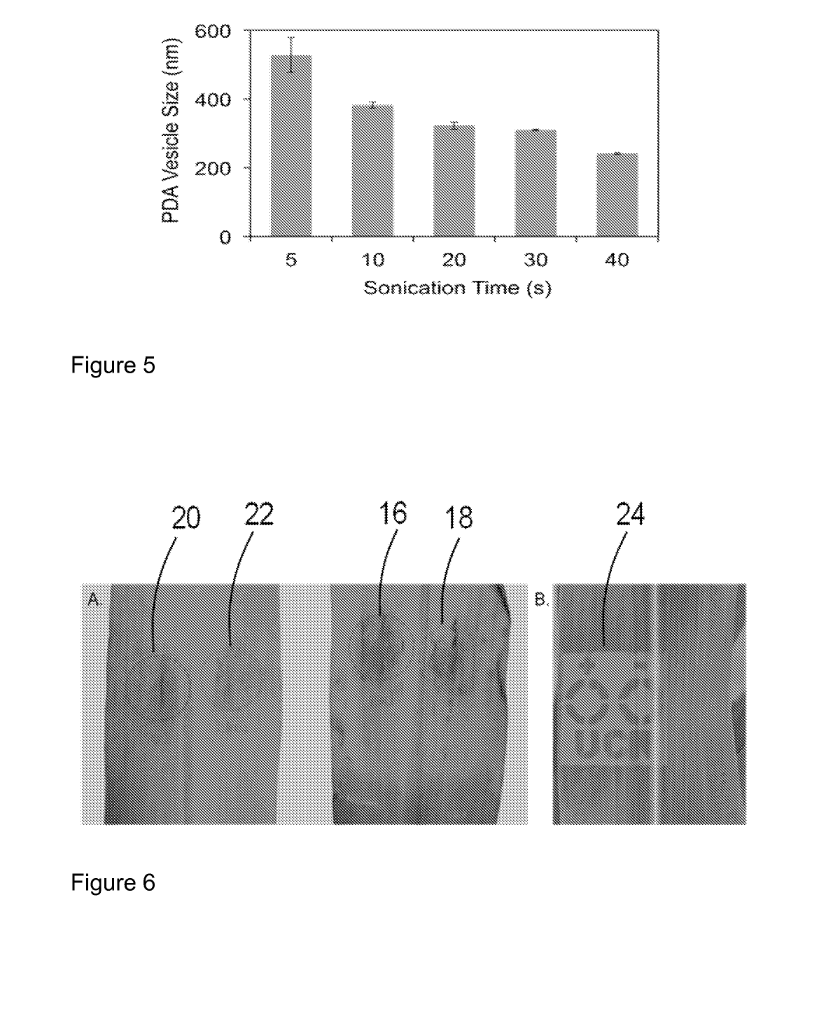

PDA liposome size as a function of sonication time is shown in FIG. 5.

### Example 3

### Visibility of Bioreagents in Maize Leaves

A method to enhance the visibility of colored bioreagents injected in maize leaves was demonstrated to increase naked-eye detection signal. By masking sections of the leaf to prevent photosynthesis, artificial chlorosis, or yellowing, of that section was induced. With chlorosis, the contrast of injected food dye was significantly enhanced (FIG. 6). A patterned mask can be used to define test and control sections.

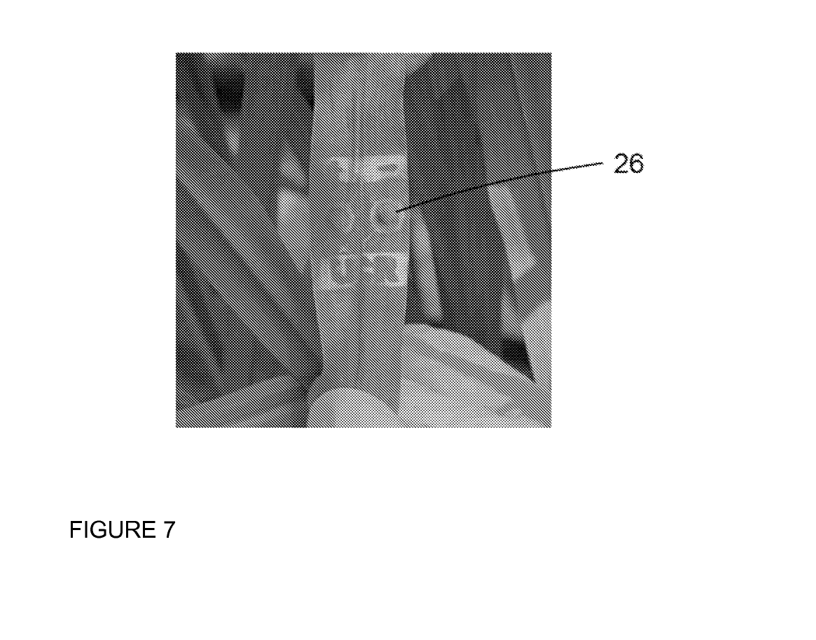

Artificial chlorosis (leaf yellowing) was induced by covering a portion of the leaf from exposure to sunlight. The contrast of injected dyes in leaves was greatly enhanced in leaf sections with artificial chlorosis as compared to dyes injected in normal leaf sections. As shown in FIG. 6A, the red dye sample 16 and blue dye sample 18 in a chlorotic leaf section is enhanced over the red dye sample 20 and blue dye sample 22 in a normal leaf section. As shown in FIG. 6B, artificial chlorosis can be induced through a mask to create patterns 24 on the leaf epidermis. In FIG. 7, enhanced visibility of a blue colored injected dye sample 26 is shown by mask patterning.

### Example 4

Development of PDA-based platform for in vivo detection of model maize pathogen, P. stewartii, a bacterium that is known to occupy the interstitial spaces in leaves [30].

### Methods

### Plant Infection

Ten-day-old maize seedlings will be inoculated by applying an inoculum of P. Stewartii containing 5×10⁷ cfu/mL over a wound created by scratching the stem.

### Synthesis of DA-NHS

Conversion of diacetylene (DA) monomers to a succinimide ester is carried out by mixing N-(3-dimethylaminopropyl)-N′-ethylcarbodiimide hydrochloride (EDC-HCl) and N-hydroxysuccinimide (NHS) in methylene chloride. The solution is stirred with a magnetic stirrer for 2 h at room temperature. The solvent is evaporated by nitrogen air stream and the residue is purified by extraction with ethyl acetate to yield DA-NHS monomers as a white solid; ¹H NMR (400 MHz, CDCl₃, δ): 0.88 (t, 3H), 1.24-1.78 (m, 28H), 2.24 (t, 4H), 2.60 (t, 2H), 2.84 (s, 4H).

### Synthesis of Liposomes and Conjugation of Aptamer

Composite PDA liposomes of DA (commercially available 10, 12 tricosadiynoic acid (TCDA) and/or 10, 12 pentacosadiynoic acid (PCDA)), DA-NHS, and phospholipid (1,2-dimyristoyl-sn-glycero-3-phosphocholine (DMPC), 1,2-dimyristoyl-sn-glycero-3-phosphoethanolamine (DMPE), and/or 1,2-dimyristoyl-sn-glycero-3-phosphoglycerol (DMPG)) are prepared by suspending monomers in deionized water followed by sonication at 80° C. (glass transition, Tg˜60° C.). Subsequent conjugation of aminated aptamer is performed by spontaneous reaction via mixing of aminated aptamer and liposomes in aqueous solution for 2 h at room temperature. Upon 254 nm UV irradiation, the liposome solution turns from opaque to deep blue. P. Stewartii aptamers are available for custom order through commercial vendors such as IBA Life Sciences (Olivette, Mo., USA) and Aptagen (Jacobus, Pa., USA).

### Methods to Optimize Liposome Composites

Liposome sensors are optimized by varying the percentage (by total lipid) of DA-aptamer, TCDA monomers, PCDA monomers and phospholipid dopants. The ratio of TCDA/PCDA monomers is known to affect the stability of color change while phospholipid doping enhances the liposome surface properties for higher sensitivity. Additional strategies used include the variation of linker length between the DAs and the conjugated aptamer, and/or the exchange of detection probe for antibodies, proteins, or peptides. The encapsulation of liposomes in hydrogel capsules can also be used to enhance their sensitivity.

### Strategy

P. stewartii-specific antibodies will be conjugated to DA monomers (DA-Ab) through a DA-NHS intermediate similar to that described above. A mixed solution of DA-Ab, unmodified DA, and DMPE in deionized water will be sonicated at 80° C. to form self-assembled PDA liposomes of mixed composition. Photopolymerization under 254 nm UV light will yield blue P. stewartii-sensing liposomes.

Alternatively, P. stewartii-specific PDA liposomes prepared similar to that describe above will be used.

NMR will confirm the synthesis of DA-NHS and fluorescence staining will confirm the subsequent DA-Ab conjugate. Dynamic light scattering (DLS) and SEM will be used to observe the size and morphology of the liposomes, respectively. The optical properties of the liposomes will be characterized through absorbance measurements by a spectrophotometer.

The sensitivity of liposomes will be systematically tested in PBS solutions containing heat-destroyed P. stewartii ranging from 10⁷ to 0 cfu/mL. The percentage of DA-Ab in the liposomes will be varied, and liposomes with average DA-Ab percentage correlating to the highest sensitivity will be selected. Selected liposomes will be further tested in excised leaves of maize plants artificially infected with P. stewartii as previously described [30]. In short, the outer leaves of maize plants will be cut and the wound sites inoculated with P. stewartii bacterial culture. After two weeks, uniform excisions of the contaminated leaves will be suspended in deionized water, and the suspension used for liposomes testing. Finally, the specificity of the strip will be evaluated against at least five other bacterial strains. For example, six samples will be tested under each of the defined conditions.

The color transitions of liposomes will be analyzed through absorbance data measured by a spectrophotometer. Specifically, the CR will be calculated [13]. Briefly, the percent blue (PB), is first defined as:

$$PB = \frac{{A}_{blue}}{{A}_{blue} + {A}_{red}} \times 100 \%$$

where A_{blue} is determined by absorbance peaks in the 620-640 nm range (PDA blue form) and A_{red} is determined by absorbance peaks in the 490-540 nm range (PDA red form). Then, the CR is defined as:

$$CR = \frac{( {PB}_{0} - {PB}_{f} )}{{PB}_{0}} \times 100 \%$$

where PB₀ is the initial blue percent of untested liposomes and PB_{f} is the final blue percent of tested liposomes. The detection limit of the liposomes will be determined as the lowest concentration of P. stewartii that induces a CR above 15%.

Detection of P. stewartii will be demonstrated in a susceptible sweet corn variety, Zea mays var. Jubilee. Ten-day-old seedlings will be inoculated by applying an inoculum containing 5×10⁷ cfu/mL over a wound created by scratching the stem [32]. Three days post-inoculation, P. stewartii-sensing liposomes will be injected using a needless syringe and incubated for one day prior to analysis. The PDA liposomes can be conjugated with P. stewartii specific antibodies, or to P. stewartii specific peptide or oligonucleotide aptamers.

Images of the detection region will be captured by a camera under uniform lighting conditions. RGB data from recorded images will be extracted using ImageJ software. The same leaves will be analyzed by ELISA and qPCR to identify levels of P. stewartii in the sample leaves. For example, twenty plants will be tested for each trial, and trials will be repeated independently three times.

The color transitions of the injected liposomes will be analyzed through red chromatic response (RCS) through extracted red-green-blue (RGB) values from color images of the PDA material in the plant leaves [33]. Further characterization of the data will be carried out by principal component analysis (PCA), which is a mathematical transformation of multivariate data that is often used to statistically visualize RGB data sets due to their high dimensionality (each image is represented by three RGB values)[34-36].

PDA liposomes will be developed for P. stewartii detection, and in vivo detection by injected liposomes will occur. For PDA liposomes containing P. stewartii antibodies, the antibodies are commercially available (available from Agdia, Inc. and Neogen Co.), or polyclonal antibodies specific to the P. stewartii acyl homoserine lactone (AHL) or exopolysaccharide (EPS) epitopes can be generated. For PDA liposomes containing P. stewartii oligonucleotide aptamers, the aptamers can be obtained as described above.

Alternative methods to improve sensor sensitivity include improving the PDA liposome composition. In this approach, sensors with DA-Ab percentages of the highest sensitivity will be subject to further optimization strategies including the variation of linker length between DA and conjugated probe, variation of total TCDA and PCDA monomers used, and doping with different phospholipids (e.g. DMPC, DMPG).

### REFERENCES

The following publications are incorporated by reference herein:
[1] Nuss, E. T., Tanumihardjo, S. A., Maize: a paramount staple crop in the context of global nutrition. Compr Rev Food Sci Food Saf. 2010, 9, 417-436.
[2] Farre, G., Twyman, R. M., Zhu, C., Capell, T., Christou, P., Nutritionally enhanced crops and food security: scientific achievements versus political expediency. Curr. Opin. Biotechnol. 2011, 22, 245-251.
[3] Smale, M., Byerlee, D., Jayne, T., Maize revolutions in sub-Saharan Africa. World Bank. 2011.
[4] Mumford, R., Boonham, N., Tomlinson, J., & Barker, I, Advances in molecular phytodiagnostics—new solutions for old problems. Eur. J. Plant Pathol. 2006, 116, 1-19.
[5] Tomlinson, J. A., Dickinson, M. J., Boonham, N., Rapid detection of Phytophthora ramorum and P. kernoviae by two-minute DNA extraction followed by isothermal amplification and amplicon detection by generic lateral flow device. Phytopathology. 2010, 100, 143-149.
[6] Mondal, K. K., & Shanmugam, V., Advancements in the diagnosis of bacterial plant pathogens: An overview. Biotechnol. Mol. Biol. Rev. 2013, 8, 1-11.
[7] Ngom, B., Guo, Y., Wang, X., Bi, D., Development and application of lateral flow test strip technology for detection of infectious agents and chemical contaminants: a review. Anal Bioanal Chem. 2010, 397, 1113-1135.
[8] Chen, Q., Lai, H., Hurtado, J., Stahnke, J., Leuzinger, K., et al., Agroinfiltration as an Effective and Scalable Strategy of Gene Delivery for Production of Pharmaceutical Proteins. Adv Tech Biol Med. 2013, DOI:10.4172/atbm.1000103
[9] Annamalai, P., Rao, A. L. N., Delivery and expression of functional viral RNA genomes in planta by agroinfiltration. Curr Protoc Microbiol. 2006, DOI:10.1002/9780471729259.mc16b02s01
[10] Wroblewski, T., Tomczak, A., Michelmore, R., Optimization of Agrobacterium-mediated transient assays of gene expression in lettuce, tomato and Arabidopsis. Plant Biotechnol J. 2005, 3, 259-273.
[11] Santos-Rosa, M., Poutaraud, A., Merdinoglu, D., Mestre, P., Development of a transient expression system in grapevine via agro-infiltration. Plant Cell Rep. 2008, 27, 1053-1063.
[12] Kikkert, J. R., Vidal, J. R., Reisch, B. I., Stable transformation of plant cells by particle bombardment/biolistics. Transgenic Plants: Methods and Protocols. 2004, 286, 61-78
[13] Wolf, S., Lucas, W. J., Deom, C. M., Beachy, R. N., Movement protein of tobacco mosaic virus modifies plasmodesmatal size exclusion limit. Science, 1989, 246, 377-379.
[14] Jelinek, R., Ritenberg, M., Polydiacetylenes—recent molecular advances and applications. RSC Adv. 2013, 3, 21192-21201.
[15] Jung, Y. K., Kim, T. W., Park, H. G., Soh, H. T., Specific Colorimetric Detection of Proteins Using Bidentate Aptamer-Conjugated Polydiacetylene (PDA) Liposomes. Adv Funct Mater. 2010, 20, 3092-3097.
[16] Armbruster, D. A., Pry, T., Limit of blank, limit of detection and limit of quantitation. Clin Biochem Rev. 2009, 29, S49-52.
[17] Lopez-Ochoa, et al., J Virol. 2006 June; 80(12):5841-53.
[18] Crawford, et al., Brief Funct Genomic Proteomic. 2003 April; 2(1):72-9.
[19] M. J. Roberts, D. E. Schimmelpfennig, E. Ashley, M. J. Livingston, M. Ash, U. Vasavada, The value of plant disease early-warning systems: a case study of USDA's soybean rust coordinated framework, United States Department of Agriculture, Economic Research Service 2006.
[20] S. Sankaran, A. Mishra, R. Ehsani, C. Davis, A review of advanced techniques for detecting plant diseases, Comput Electron Agr, 72(2010) 1-13.
[21] J. Tomlinson, M. Dickinson, E. Hobden, S. Robinson, P. Giltrap, N. Boonham, A five-minute DNA extraction method for expedited detection of Phytophthora ramorum following prescreening using Phytophthora spp. lateral flow devices, Journal of microbiological methods, 81(2010) 116-20.
[22] C. Guo, S. Liu, Z. Dai, C. Jiang, W. Li, Polydiacetylene vesicles as a novel drug sustained-release system, Colloids and Surfaces B: Biointerfaces, 76(2010) 362-5.
[23] C. Guo, S. Liu, C. Jiang, W. Li, Z. Dai, H. Fritz, et al., A promising drug controlled-release system based on diacetylene/phospholipid polymerized vesicles, Langmuir, 25(2009) 13114-9.
[24] D. H. Charych, J. O. Nagy, W. Spevak, M. D. Bednarski, Direct colorimetric detection of a receptor-ligand interaction by a polymerized bilayer assembly, Science, 261(1993) 585-8.
[25] D. H. Charych, W. Spevak, J. O. Nagy, M. D. Bednarski, Specific interaction of influenza-virus with organized assemblies of polydiacetylenes, MRS Proceedings, 292(1993) 153-61.
[26] Y. K. Jung, T. W. Kim, H. G. Park, H. T. Soh, Specific Colorimetric Detection of Proteins Using Bidentate Aptamer-Conjugated Polydiacetylene (PDA) Liposomes, Advanced Functional Materials, 20(2010) 3092-7.
[27] J. T. Wen, C. Castro, H. Tsutsui, In Planta Microsphere-Based Lateral Flow Leaf Biosensor in Maize, Journal of laboratory automation, (2014) 2211068214551826.
[28] Q. Chen, H. Lai, J. Hurtado, J. Stahnke, K. Leuzinger, M. Dent, Agroinfiltration as an effective and scalable strategy of gene delivery for production of pharmaceutical proteins, Advanced techniques in biology & medicine, 1(2013).
[29] D. A. Armbruster, T. Pry, Limit of blank, limit of detection and limit of quantitation, The Clinical Biochemist Reviews, 29(2008) S49.
[30] S. P. Thapa, D. H. Park, C. Wilson, J. H. Hur, C. K. Lim, Multiplex PCR assay for the detection of Pantoea stewartii subsp. stewartii using species-specific genetic markers, Australasian Plant Pathology, 41(2012) 559-64.
[31] S. Okada, S. Peng, W. Spevak, D. Charych, Color and chromism of polydiacetylene vesicles, Accounts Chem Res, 31(1998) 229-39.
[32] C. M. Herrera, M. D. Koutsoudis, X. Wang, S. B. von Bodman, Pantoea stewartii subsp. stewartii exhibits surface motility, which is a critical aspect of Stewart's wilt disease development on maize, Molecular plant-microbe interactions: MPMI, 21(2008) 1359-70.
[33] Volinsky, R., Kliger, M., Sheynis, T., Kolusheva, S., Jelinek, R., Glass-supported lipid/polydiacetylene films for colour sensing of membrane-active compounds. Biosens. Bioelectron. 2007, 22, 3247-3251.
[34] Li, Y., Wang, L., Yin, X., Ding, B., et al., Colorimetric Strips for Visual Lead Ion Recognition Utilizing Polydiacetylenes Embedded Nanofibers. J. Mater. Chem. A 2014.
[35] Soga, T., Jimbo, Y., Suzuki, K., Citterio, D., Inkjet-printed paper-based colorimetric sensor array for the discrimination of volatile primary amines. Anal. Chem. 2013, 85, 8973-8978.
[36] Yoon, B., Park, I. S., Shin, H., Park, H. J., et al., A Litmus-Type Colorimetric and Fluorometric Volatile Organic Compound Sensor Based on Inkjet-Printed Polydiacetylenes on Paper Substrates. Macromol. Rapid Comm. 2013, 34, 731-735.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method for detecting an analyte in a plant, comprising
injecting an analyte-capture agent into a leaf of a plant, and
in the leaf, optically detecting complexes formed by capturing an analyte with the injected analyte-capture agent.

2. The method of claim 1, wherein the analyte-capture agent comprises an analyte-interacting component connected to a microcarrier.

3. The method of claim 2, wherein the analyte-interacting component is an antibody, peptide aptamer, or oligonucleotide aptamer.

4. The method of claim 3, wherein the analyte-interacting component specifically recognizes the analyte.

5. The method of claim 2, wherein the microcarrier is a microsphere or vesicle.

6. The method of claim 5, wherein the microcarrier is a polydiacetylene liposome.

7. The method of claim 1, wherein the analyte is a metabolite or nutrient of the plant, is a plant pathogen or a component of the plant pathogen, or is an environmental toxin.

8. The method of claim 1 wherein the injecting is performed by propelling the analyte-capture agent into the leaf interstitial space.

9. The method of claim 1, wherein the optically detecting comprises colorimetric detection of the complexes after analyte capture by the injected analyte-capture agent.

10. The method of claim 1, wherein the optically detecting comprises interacting the complexes with a secondary antibody to form secondary antibody combinations, and colorimetric detection of the combinations.

11. The method of claim 1, wherein the plant is a crop plant.

12. The method of claim 1, wherein the plant is a living plant, and the leaf is attached to the plant at least until the analyte capturing.

13. The method of claim 1, wherein the detecting is performed on chlorotic leaf tissue.

14. A method for detecting an analyte in a plant, comprising
injecting an analyte-capture agent into a leaf of a plant, the analyte-capture agent comprising an analyte-interacting component connected to a polydiacetylene liposome, wherein the analyte-interacting component specifically recognizes an analyte in the plant,
forming complexes in the leaf by capturing the analyte with the injected analyte-capturing agent, and
in the leaf, visually detecting a color change in the complexes after analyte capture by the injected analyte-capture agent.

15. Method of claim 14, wherein the analyte-interacting component is an antibody, peptide aptamer, or oligonucleotide aptamer.

16. Method of claim 14, wherein the analyte is a pathogen of the plant or is a component of the plant pathogen.

17. The method of claim 16, wherein the plant is a living plant, and the leaf is attached to the plant at least until the analyte capture.

18. The method of claim 17, wherein the plant is a crop plant.

19. The method of claim 14, wherein the detecting is performed on chlorotic leaf tissue.
